(19) United States
(12) Patent Application Publication
(10) Pub. No.: US 2011/0238140 A1
(43) Pub. Date: Sep. 29, 2011

(54) LIGHT EXPOSURE DEVICE FOR TREATING A LIVING ORGANISM'S BODY

(76) Inventor: Abdula KURKAYEV, Budapest (HU)

(21) Appl. No.: 13/019,996

(22) Filed: Feb. 2, 2011

(30) Foreign Application Priority Data
Mar. 25, 2010 (AT) GM197/2010

(51) Int. Cl. A61N 5/067; A61N 5/06

(52) U.S. Cl. 607/89; 607/90

(57) ABSTRACT

A light exposure device for treating a living organism's body including controllable light sources (12) and a central control system (I) of light sources (12). Pursuant to one of embodiments, light sources (12) are arranged into groups (IV) and each group (IV) of light sources (12) is coupled to central control system (I) enabling an independent operation of each group (IV) of light sources (12). The device also includes a system (II) for measuring physiological parameters of a living organism and a physiological parameters controller (V). The measuring system (II) and controller (V) are connected to central control system (I).

### BACKGROUND OF THE INVENTION

1. Field of the Invention

The invention relates to a light exposure device for treating a living organism's body, comprising controllable light sources and a central control system for controlling the light sources. The invention is applicable in medical techniques, especially in the field of laser devices where different modes of light exposure are used to exert a therapeutic effect onto a human or animal body for diverse purposes.

2. Description of the Prior Art

A method of photo-dynamic therapy is known from the literature (Department of pathology, Norwegian Radium Hospital, University of Oslo, 1997, Jan. 15, 79 (12), 2282-308). The known method comprises application of the photosensitizer 5-aminolevulinic acid prior to laser light irradiation and is used for treatment of superficial ulcers, mouth mucous tunic etc.

Radio frequency exposure devices for human application are commercially available (e.g. EHY-2000 by Oncotherm Kft., Hungary). Available devices comprise radio wave sources mounted on a movable frame, wherein the radio frequency exposure is limited to a narrow site of the targeted treatment area. It is a disadvantage that extra time and effort are necessary to irradiate a larger target area or several target areas.

A portable laser device for exerting light influence on a human skin is known from RU 2 291 725 dd. 20.01.2007. The known embodiment consists of at least two laser light sources, a control unit for the laser light sources which enables independent functioning of several of the laser light sources, and an optical instrument for bundling laser light rays irradiated by laser light sources, which reshapes every bundle in a certain way enabling different forms of spot lights. This portable laser device also enables different modes of laser light irradiation providing for lower power consumption.

It is a disadvantage of the known laser device that it shows just a limited therapeutic effect for lack of a precise management of the irradiation dose parameters including the radiating power, time and area of irradiation.

Another device for photo-dynamic therapy (PDT) is known from RU 86432 dd. 10.09.2009. This device comprises a control unit connected to a power supply, an oscillator consisting of several light diodes, and a cooling unit mounted on the inner surface of the cover, wherein the control unit enables operating every light diode independently and obtaining different irradiation patterns and spectra.

RU No. 46435 dd. 10.07.2005 describes a medical laser device comprising a power supply and a microprocessor-based control unit connected to the power supply, wherein the control unit is also coupled to an optical instrument. The optical instrument contains semiconductor laser light sources and a light guide. The control unit enables stabilizing a wavelength of laser light, monitoring a temperature of the targeted treatment area, and is provided with warning lights and alarm sounds.

An object of the present invention is to provide an effective and universally applicable light exposure device suitable for treatment of different disorders of a living organism.

### SUMMARY OF THE INVENTION

The invention is based on the insight that the central control system secures operation of a light source controller and of a system for measuring physiologic parameters, wherein light sources are arranged into groups and each group of light sources is coupled to the controller, enabling controlled operation of each group of light sources.

The device according to the present invention enables faster wound healing, lowers edema and stopping painful sensations of different etiology, among other uses and also enables successful postoperative treatment after liposuction. The device can also be used to abate inflammations of different types, as well as to rehabilitate damaged muscles and sinews etc.

### BRIEF DESCRIPTION OF THE DRAWING

FIG. 1 represents a schematic depiction of an embodiment of the present light exposure device for treating a living organism's tissues.

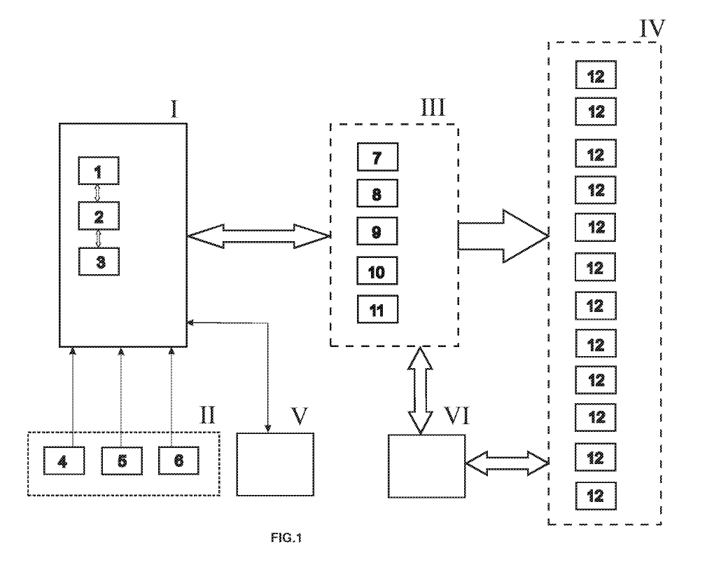

### DETAILED DESCRIPTION OF THE PREFERRED EMBODIMENTS

One of the embodiments of the present invention causes neither a detectible increase in temperature of the treated tissues, nor macroscopically visible changes in the tissue structure. Hence, neither the treated nor the surrounding healthy tissues are heated and therefore are not thermally damaged.

In several cases, it is necessary to expose diverse organs of a living organism to light pulses of different types.

Efficacy of light exposure is increased through harmonizing light irradiation in accordance with the individual physical condition of a treatable living organism. Arranging light sources into groups and controlling each group of light sources enables optimization of the luminous radiation guided to target areas. Light sources are operated irrespectively of each other. However, a simultaneous control of all groups of light sources at their highest level of radiating power is also possible. A system for measuring physiological parameters of a treatable living organism enables synchronizing irradiation modes of light sources in accordance with the measured physiologic parameters. Physiological parameters of a treatable living organism are preferably continuously checked during a light exposure session whereby the central control system is able to maintain optimal operation parameters for controlling radiating power of each group of light sources in accordance with individual measured physiological parameters.

The system for measuring physiologic parameters comprises e.g. a pulse sensor and/or a temperature sensor and/or at least one sensor for measuring brain frequencies.

Light sources are preferably controllable light sources selected from the group containing light sources of high power and large range of wavelength, single LEDs, LEDs arranged in a matrix, halogen light sources, laser diodes and laser light sources. The radiating power of a light source is preferably in the range between 1 mW and 10 W. In a pulse mode, at a pulse frequency from 0 to 10 kHz and pulse duration from 100 ns to 100 ms, peak radiating power amounts e.g. to 1 W to 10 kW. Light sources are used to irradiate light pulses of pulse duration preferably in the range from 100 ns to 10 ms.

Pursuant to one of embodiments of the present invention, light sources are fixed on frame walls.

Pursuant to one of embodiments of the present invention, the cover is made in the form of a hull, which at least partially envelops a treatable body.

Preferably, a cooler is used to cool down the light sources. The cooler is connected to the light source controller, which enables further utilization of surplus heat removed from the light sources.

Light exposure device comprises a central control system I, a system II for measuring physiological parameters of a treatable living organism, a system III for input and control of operation parameters; groups IV of light sources 12, a controller V and a cooler VI for cooling down light sources 12.

Central control system I comprises a data input system 1, into which an operator feeds necessary operation parameters, a data saver 2 and a synchronizer 3 for harmonizing physiological parameters of a treated living organism to irradiation modes and irradiation parameters of light sources 12.

Central control system I is connected to system II for measuring physiological parameters of a living organism. Measuring system II enables continuously monitoring physiological parameters and comprises e.g. at least one sensor 4 for measuring brain frequencies (EEG), a pulse sensor 5, or a temperature sensor 6.

System III for input and control of operation parameters comprises a radiating power controller 7 for groups IV of light sources 12, a controller 8 for intensity of current supplied to light sources 12, a frequency modulation controller 9, a module 10 for controlling and inputting run-time of light sources 12 and a pulse mode unit 11 of groups IV of light sources 12.

Groups IV of light sources 12 consist of groups of single light sources 12, preferably placed on the hull.

Controller V, connected to central control system I, enables continuous monitoring of the condition of a living organism during an irradiation session. Central control system I ensures selecting an optimal irradiation mode of light sources 12 in accordance with individual physiological parameters of a living organism.

Cooler VI cools down groups IV of light sources 12 or removes their excess heat during irradiation session, respectively.

Into data input system 1, an operator feeds operation parameters in accordance with indicators of physiological parameters of an irradiated body. Central control system I corrects by means of synchronizer 3 data fed in accordance with physiological parameters of a living organism measured by measuring system II. System III for input and control of operational parameters determinates and checks operational parameters of light emitted by each group IV of light sources 12. While operating in pulse mode, unit 11 determines operational parameters and functioning of group IV of light sources 12.

We claim:

1. A light exposure device for therapeutic treatment of a living organism's body comprising controllable light sources and a central control system for the light sources, wherein the light sources are arranged into groups and each group of light sources is coupled to the central control system in a way that enables independent operation of each said group of light sources, and a system for measuring physiological parameters of a living organism and a physiological parameters controller, wherein the measuring system and the controller are connected to the central control system.

2. The device as claimed in claim 1, wherein the measuring system for measuring physiological parameters comprises a pulse sensor and/or a temperature sensor and/or at least one sensor for measuring brain frequencies.

3. The device according to claim 1, wherein the light sources are controllable light sources selected from the group including light sources of high power and a large range of wavelength, single LEDs, LEDs arranged in a matrix, halogen light sources, laser diodes and laser light sources.

4. The device according to claim 2, wherein the light sources are controllable light sources selected from the group including light sources of high power and a large range of wavelength, single LEDs, LEDs arranged in a matrix, halogen light sources, laser diodes and laser light sources.

5. The device according to claim 1, wherein the light sources are used to emit light pulses having a pulse duration in the range of 100 ns and 100 ms.

6. The device according to claim 2, wherein the light sources are used to emit light pulses having a pulse duration in the range of 100 ns and 100 ms.

7. The device according to claim 3, wherein the light sources are used to emit light pulses having a pulse duration in the range of 100 ns and 100 ms.

8. The device according to claim 4, wherein the light sources are used to emit light pulses having a pulse duration in the range of 100 ns and 100 ms.

9. The device according to claim 1, wherein the device further comprises a cover and the light sources are fixed on walls of the cover.

10. The device according to claim 2, wherein the device further comprises a cover and the light sources are fixed on walls of the cover.

11. The device according to claim 3, wherein the device further comprises a cover and the light sources are fixed on walls of the cover.

12. The device according to claim 5, wherein the device further comprises a cover and the light sources are fixed on walls of the cover.

13. The device according to claim 9, wherein the cover is made in a form of a hull, which at least partially envelops a treatable body.

14. The device according to claim 10, wherein the cover is made in a form of a hull, which at least partially envelops a treatable body.

15. The device according to claim 11, wherein the cover is made in a form of a hull, which at least partially envelops a treatable body.

16. The device according to claim 12, wherein the cover is made in a form of a hull, which at least partially envelops a treatable body.

17. The device according to claim 1, further comprising a cooler used to cool down laser light sources.

18. The device according to claim 2, further comprising a cooler used to cool down laser light sources.

19. The device according to claim 3, further comprising a cooler used to cool down laser light sources.

20. The device according to claim 5, further comprising a cooler used to cool down laser light sources.
